(19) United States
(12) Patent Application Publication
(10) Pub. No.: US 2023/0123857 A1
(43) Pub. Date: Apr. 20, 2023

(54) Process of Using a Fish Plasma Component in a Nutrient Medium for Cell Culture

(71) Applicant: Salmonics LLC, Brunswick, ME (US)

(72) Inventors: Evelyn S. Sawyer, Freeport, ME (US); Paul A. Janmey, Media, PA (US)

(73) Assignee: Salmonics LLC, Brunswick, ME (US)

(21) Appl. No.: 17/463,690

(22) Filed: Sep. 1, 2021

(51) Int. Cl. C12N 5/0793; C12N 5/07; C12N 5/0797; G01N 33/50; G01N 33/579; A61D 99/00

(52) U.S. Cl. CPC C12N 5/0619; C12N 5/0601; C12N 5/0623; G01N 33/5002; G01N 33/579; A61D 99/00; G01N 2400/50; C12N 2500/14; C12N 2500/36; G01N 2011/0026

(57) ABSTRACT

A process of using a fish plasma component in a nutrient medium for cell culture includes obtaining a fish that is a progeny of domesticated broodstock that are reared under consistent and reproducible conditions. Blood is obtained from the fish, and plasma is separated from the blood. One or more specific components of the plasma are then extracted, and cells are cultured in a nutrient medium using the one or more extracted plasma components, and none of any remainder of the plasma. The plasma and/or the plasma components is/are tested for presence and/or level of endotoxin. Extracting the one or more specific components of the plasma, and/or culturing the cells is only performed if the testing indicates an endotoxin level below a predetermined threshold. The cells cultured using the extracted one or more plasma components are other than fish cells.

### FIELD OF THE INVENTION

The invention relates generally to the culture of tissue, including cells and organs, and more specifically to the culture of tissue using at least one component of plasma derived from fish.

### BACKGROUND OF THE INVENTION

Tissue culture, the production of living tissue in vitro, permits numerous applications that would be difficult or impossible in a living organism. These applications include in vitro applications such as diagnosing disease and assessing toxicity, and more recently, the production of therapeutics, including vaccines and recombinant proteins, and growing human tissue, including cells and organs, for therapeutic applications (tissue engineering).

The culture of animal tissue usually requires animal biologics: either whole serum, most commonly fetal calf serum (FBS), or plasma components, for “serum-free” media or biological gels. Current methods for deriving mammalian serum or plasma components are well-known. The raw material is human or bovine blood from which the cellular portion is removed by centrifugation. If an anticoagulant is used, the liquid portion is plasma; if the blood is allowed to clot, the liquid portion is serum. The most widely used method of fractionating human or bovine plasma is the Cohn process (Cohn et al., 1946), which utilizes adjustments of temperature, pH, and ethanol to separate plasma proteins.

However, the risk of the presence of mammalian infectious organisms in mammalian plasma or serum products used in tissue culture for therapeutics is an increasing concern. Some plasma proteins can be manufactured by recombinant technology, although others, especially the glycoproteins, must be obtained from humans or animals. Although various viral-inactivation treatments for plasma or serum components are frequently used, problems remain in achieving 100% inactivation without compromising quality. An even more serious concern is the emergence of transmissible spongiform encephalopathies (TSEs) such as “mad cow disease”, and the possibility of prions or infectious proteins in plasma or serum derivatives. The later problem is especially difficult, since at present, it is not possible to predict which individual blood donors, human or bovine, might years later develop a prion disease.

In order to improve the safety profile of animal products used in mammalian cell culture, Sawyer et al. (U.S. Pat. Nos. 5,426,045 and 5,443,984) developed a method using fish whole serum to replace FBS or other animal serum. This fish serum provided the important advantage of a low probability of mammalian infectious agents, and successfully replaced FBS by promoting growth in a few cell lines. However, it was toxic to many mammalian cells, and ineffective for others.

Sawyer et al., in the '045 patent, identified (among several factors) the high lipid content of fish serum as “potentially inhibiting” to mammalian cell growth. Therefore, we attempted to overcome the toxicity problem by removing some of the lipid.

Using known methods (Condie, 1979: Ando, 1996), lipids and lipoproteins were separated from the plasma of Atlantic salmon (Salmo salar). The delipidated plasma was used to replace FBS on several mammalian cell lines. In each case, the material proved toxic to the mammalian cells.

This toxicity pointed to a similar problem with the removed lipid. Furthermore, cell culture teaches a like-to-like match or species-specificity of biological materials used and cells being cultured (Hewlett, 1991). Because fish lipids are significantly different from mammalian lipids (Rabin and Vernier, 1989), it seemed unlikely that the fish lipid would promote mammalian cell growth. Nonetheless, the salmon lipid was tried as a media supplement for a mammalian cell line (Vero). The unexpected result was enhanced growth of the mammalian cells.

Because of the success of the lipid component, other components, in particular, plasma proteins, which might be useful in mammalian tissue culture, were separated (purified) from the whole plasma to attempt to overcome whole plasma toxicity. This approach presented the problem of dissimilar structure between fish and mammalian plasma proteins, and therefore a low probability that a given protein would function in a similar manner to its mammalian homologue. Doolittle (1987) studied fish plasma proteins from the perspective of comparative physiology and evolution, and found only partial identity in amino acid sequence to their mammalian homologues. For example, lamprey fibrinogen is less than 50% homologous to human fibrinogen, and salmon transferrin has only a 40-44% amino acid sequence identity with human transferrin (Denovan-Wright, 1996). This and similar data on percent homology for other plasma proteins such as fish albumin (28% homology) would dissuade those skilled in mammalian cell culture from attempting to use the fish homologue.

Additional difficulties were encountered because the usual method of fractionating mammalian plasma protein (Cohn et al., 1946) could not be used with salmon plasma. The Cohn process is the most widely used method of separating, or fractionating, serum or plasma into its components. Although this process has been improved and modified considerably, it achieves basic separation and precipitation of plasma fractions by cold temperature, and adjustments in pH and ethanol concentration. Because the temperature of salmon blood is often 4° C. or less when it is drawn from the fish during winter, temperature separation of proteins was not a consistent or reliable method.

Sawyer et al. (U.S. Pat. No. 6,007,811) extracted two proteins, fibrinogen and thrombin, from salmon plasma for use as a sealant for hemostasis. However, immunoblots and SDS-PAGE showed a different primary structure for human (lane 1) vs. salmon (lane 2) fibrinogen (FIG. 1). Furthermore, this application is unrelated to cell culture, and provided no indication that these proteins would be less cytotoxic than the salmon whole plasma.

Fibrinogen and thrombin form a fibrin gel, and an optimal environment for certain mammalian cells, especially neurons, is a three-dimensional matrix, usually a gel made from mammalian proteins. Methods known for mammalian plasma were used to purify fibrinogen and thrombin from salmon plasma. Mouse spinal cord neurons were chosen as test cells for the fish fibrin gel, because they are a model for human neuron regeneration, and are very sensitive to toxicity.

When the survival and process outgrowth of these neurons was compared in human, bovine, and salmon fibrin gels, the unexpected result was the superior performance of the neurons in the fish material. Since mammalian fibrin gels are already being used to grow neurons for therapeutic purposes, the improved neuron process outgrowth and safety profile of the fish gels would make them an attractive alternative. Additional advantages of the salmon gel were its ease of preparation (lyophilized salmon fibrinogen can be resolublized at room temperature instead of at 37° C.), and resistance to changes in pH and osmolality (FIG. 2).

The latter developments were disclosed in U.S. Pat. No. 6,599,740, which issued on Jul. 29, 2003, and U.S. Pat. No. 6,861,255, which issued on Mar. 1, 2005, both to Sawyer et al. The material presented in Sawyer et al., 2003 and 2005 has led to many successful studies, both in in vitro and in vivo (Smith et al., 2016; Sharpe et al., 2012; Zhu et al., 2017). However, some researchers reported unexplained mixed results with certain batches or lots. Experience in preparing the fish plasma components and the similar experience of other researchers since these patents issued, has led to the conclusion that certain improvements are needed.

Sawyer et al., 2003 and 2005 are similar in addressing the use of fish plasma proteins and lipids to culture mammalian cell or tissues. Both use the words “cells” and “tissue” interchangeably although “tissue” refers to a group of cells that perform the same function. Both patents emphasize the importance of cold temperatures of 4° C. or lower during processing, but do not address important steps for freezing and thawing plasma, or critical testing of the fish plasma proteins. The two patents differ by the focus in Sawyer et al., 2005 on culture of mammalian stem cells and tissue.

Overall, modifications to freezing and thawing of plasma, processing temperature of plasma proteins, and critical chemical and physical testing of extracted proteins, would lead to significant improvements in protein yield and quality.

### 1. Temperature

### A. Plasma Freezing

Because most fish plasma is available only when fish are killed for market, the plasma destined for extraction of plasma proteins or lipids is usually frozen for later laboratory processing. Both Sawyer et al., 2003 and 2005 teach the addition of an antioxidant to the plasma before extraction of components, but only plasma to be used for lipids is frozen at −80° C., whereas home freezers typically reach only a temperature of −20° or so. The addition of this freezing step for plasma proteins would prevent their degradation especially in long term storage, and improve protein yield (Myllya, 1991),

### B. Processing

Sawyer et al., 2003 and 2005 state: “The essential requirements are that all process temperatures remain at or below 4° C.” This is the optimum practice for lipids, but for thawing frozen plasma destined for extraction of proteins such as fibrinogen this practice often results in the formation of cryoprecipitate. Cryoprecipitate is a concentration of cold-insoluble high molecular weight proteins (especially fibrinogen) that occurs during thawing. In fact, Rose et al. 1990, in an effort to produce cryoprecipitate, teach raising the temperature of thawing plasma “preferably to 4° C.” . Much of the fibrinogen is then lost in the remaining plasma. This problem can be solved by briefly raising the plasma thawing temperature closer to 5-6° C., which allows the fibrinogen to go back into solution and process yields to improve (Rock, 1991).

### 2. Endotoxin Testing

Both Sawyer et al. patents state that “there must be no cyotoxic chemical residues in the plasma”, but make no mention of testing for endotoxin, a common cytotoxic chemical contaminant. This toxin is released by Gram-negative bacteria that are abundant in the marine environment and therefore likely to be present in marine fish plasma. Even very low levels of endotoxin are cytotoxic, and most suppliers guarantee levels of less than 1 endotoxin unit/ml in their products (Swartz et al., 2014).

Endotoxin is not mentioned in Sawyer et al., 2003 and 2005, and testing for this toxic chemical using the Limulus Amebocyte Lysate assay or similar assay could prevent failure of salmon proteins and lipids to grow and maintain mammalian cells.

### 3. Rheometry

Cells respond to both chemical and mechanical stimuli, and when a salmon fibrin gel composed of or including fibrinogen and thrombin is used as a substrate for mammalian cell culture, the mechanical properties of that gel are important. Rheometry of a fibrin gel is necessary to determine the property of sheer modulus or stiffness, typically as measured in Pascals.

Sawyer et al., 2003 and 2005 do not mention the need to test the sheer modulus or stiffness of a fibrin gel composed of salmon fibrinogen and thrombin. For many applications, this should exceed 1000 Pascals (Pa) to allow dilution, but this measure of clot stiffness is variable in the culture of mammalian tissue. For example, Georges et al., 2006 found that neurons prefer a soft (˜50 Pa) salmon fibrin gel, and astrocyte spread and adherence is greater on stiffer gels. FIG. 3 shows variation in stiffness of several lots of salmon gels formed by polymerizing salmon fibrinogen with salmon thrombin including unusable gels made with two different lots of fibrinogen (#1545 and 1545T). Polymerization was initiated by adding 2 μL salmon thrombin (10 IU/ml) to 250 μL fibrinogen (20 mgs/ml). Rheology was performed 24 hours after rehydration of lyophilized proteins in a Kinexus rheometer 2% strain, 1 Hertz for 1000 seconds. Because all the gels in FIG. 3 polymerize and appear normal, the failing gels could not be detected without the use of rheometry.

Failure of a salmon fibrin gel relates to the batch or lot of fibrinogen used, and can be caused by many factors including the health status of the fish, process temperatures above 6° C., partial activation of proteases, and many unknown factors. Therefore, rheometry is important before salmon fibrin gels can be used as a scaffold or substrate in mammalian cell or tissue culture.

### BRIEF SUMMARY OF THE INVENTION

According to an aspect of the invention, a process of using a fish plasma component in a nutrient medium for cell culture includes obtaining a fish that is a progeny of domesticated broodstock that are reared under consistent and reproducible conditions. Blood is obtained from the fish, and plasma is separated from the blood. One or more specific components of the plasma are then extracted, and cells are cultured in a nutrient medium using the one or more extracted plasma components, and none of any remainder of the plasma. The plasma and/or the plasma components is/are tested for presence and/or level of endotoxin. Extracting the one or more specific components of the plasma, and/or culturing the cells is only performed if the testing indicates an endotoxin level below a predetermined threshold. The cells cultured using the extracted one or more plasma components are other than fish cells.

The process can also include freezing the plasma at a temperature of −60° C. or lower after separating the plasma from the blood, and thawing the frozen plasma before extracting one or more specific components of the thawed plasma. Thawing the frozen plasma can include allowing the frozen plasma to reach a temperature at which any cryoprecipitate present in the plasma redissolves back into solution, typically at 5-6° C. or so.

The one or more specific components of the plasma can include, for example, fibrinogen and thrombin, and culturing cells using the extracted plasma components, and none of any remainder of the plasma, in a nutrient medium, can include preparing a gel with the fibrinogen, the thrombin, and calcium in tissue culture media. Culturing the cells can also include embedding the cells in the gel. The process can also include determining a sheer modulus of the gel, which can include performing rheometry on the gel. The process can also include selecting the cells cultured using the extracted one or more plasma components based at least in part on the determined sheer modulus of the gel.

The fish from which the blood is obtained preferably is larger than two kilograms and/or reared by standard husbandry methods.

Obtaining blood from the fish can include, for example, rendering the fish to a level of loss of reflex activity. Blood can be drawn, for example, from a caudal blood vessel. Obtaining blood from the fish can also include reducing the levels of proteolytic enzymes and non-protein nitrogen present in the blood of the fish, prior to rendering the fish to a level of loss of reflex activity.

Separating plasma from the blood can include centrifuging the blood.

Extracting the one or more components from the plasma can include performing an extraction process on the plasma such that no cytotoxic chemical residues remain in the one or more plasma components, and no oxidation of plasma lipids occurs.

The cells cultured using the one or more extracted plasma components can be, for example, mammalian cells, for example, neurons and/or stem cells.

The cells cultured using the one or more extracted plasma components can include organ tissue.

The cells cultured using the one or more extracted plasma components can be insect cells.

The fish can be a cold-water fish, such as, for example, a salmonid.

Culturing cells in the one or more extracted plasma components can include seeding the cells in a defined medium and the one or more extracted plasma components.

The one or more components extracted from the plasma can include lipids. The process can also include adding an antioxidant to the plasma prior to extracting the one or more components from the plasma.

The one or more components extracted from the plasma can include a plasma protein, such as, for example, transferrin, albumin, fibrinogen, fibronectin, thrombin, and/or enzymes.

### BRIEF SUMMARY OF THE DRAWING FIGURES

FIG. 1 shows an SDS-PAGE analysis of primary structures of human (lane 1) and salmon (lane 2) fibrinogen.

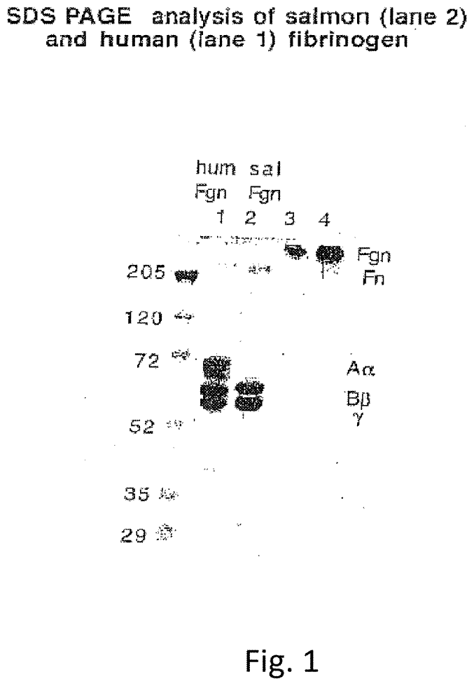

FIG. 2 illustrates the resistance to changes in pH and osmolality of salmon fibrin gel.

FIG. 3 shows a rheology of salmon fibrin gels made from four lots of salmon fibrinogen.

FIG. 4 illustrates the effect of salmon lipid on Vero cells after 48 hours.

FIG. 5 shows mammalian neurons grown in bovine fibrin gels.

FIG. 6 shows mammalian neurons grown in fish fibrin gels.

FIG. 7 is a graph depicting the difference in average total neurite length per cell of mammalian neurons grown in bovine fibrin gels and mammalian neurons grown in fish fibrin gels.

FIG. 8 shows human neural stem cells cultured in various fibrin gels.

FIG. 9 is a chart showing the number of Hoechst-stained nuclei of human neural stem cells present after six days of culturing in fibrin gels.

### DETAILED DESCRIPTION OF THE INVENTION

Because of the many risks and uncertainties inherent in human and other mammalian biologics, and the cytotoxicity and ineffectiveness of fish whole serum or plasma, the method of the present invention uses fish plasma components that are separated (purified) from the whole plasma of farmed fish, which can be used in culturing mammalian tissue. Fish species for which consistent and reproducible methods of production are well established are suited for use in the method of the present invention. Exemplary use of salmonids, specifically the Atlantic salmon (Salmo salar), will be described and demonstrated; however, the scope of the present invention is not limited to use of this particular species.

In addition to the advantage of relative safety, the substances (fractions) derived from salmon plasma enhance growth of certain mammalian cells. However, fish plasma components are not conventionally used, and actually have been discouraged for use in mammalian cell culture for several reasons, including:

1. Fish whole serum or plasma has failed to supplement or replace FBS in the media used for mammalian cell culture due to the frequent toxicity and ineffectiveness of the fish material.

2. Fish are traditionally considered to be free-ranging, wild animals. Therefore, apparent uncertainty in quality, availability, and reproducibility of their blood products would seem to make them unsuitable donors.

3. The usual, and most cost-effective, method of fractionating human or other mammalian serum or plasma proteins (Cohn process) is not suitable for salmon or other coldwater fish, because the separation depends in part on temperature effects. Because salmon plasma can vary in temperature from about 0° C. to 16° C. seasonally, this method can be unreliable.

4. Conventional cell culture teaches a like-to-like match or species-specificity of biological materials in the culture media, and cells being cultured (Hewlett, 1991). For example, Hewlett cautions against the use of lipoproteins from other than human or bovine sources for human cells due to species-specificity. Likewise, fish serum is recommended over bovine serum for the culture of (RTG2) rainbow trout gonadal cells (DeKoning and Kaattari, 1992).

5. Fish plasma proteins have been studied from the perspective of comparative physiology and evolution, and found only partially identical to their mammalian homologues (Doolittle, 1987). For example, salmon transferrin has only a 40-44% amino acid sequence identity with human transferrin (Denovan-Wright et al., 1996). This and similar data for other plasma proteins such as fish albumin (Davidson et al., 1989) would dissuade those skilled in the field of mammalian cell culture from trying fish proteins.

6. Compared to plasma from mammals, salmon and trout plasma contain oxidative enzymes that remain active at low temperatures, and therefore are likely to generate cytotoxic substances. Therefore, special preparation and handling procedures are required.

According to the method of the invention, each of the noted obstacles has been overcome, and the advantages of the use of fish plasma components are exploited.

The method of the present invention takes advantage of the fact that commercial salmon aquaculture has grown dramatically. In Maine alone, there are well over six million fish, averaging 2-5 kilograms each, reared in offshore pens annually. The availability of raw material (blood) and the efficiency of recently developed blood-drawing methods and devices contribute to a large supply and availability of fish blood. By utilizing these domesticated fish stocks reared in aquaculture facilities, plasma can be obtained with product consistency similar to plasma from herds of cattle reared for this purpose.

Further, although amino acid sequences in fish and mammalian plasma proteins have less than 50% identity, many of the critical sequences or active sites required for similar function in both fish and mammals, are highly-conserved among vertebrates including salmon and trout.

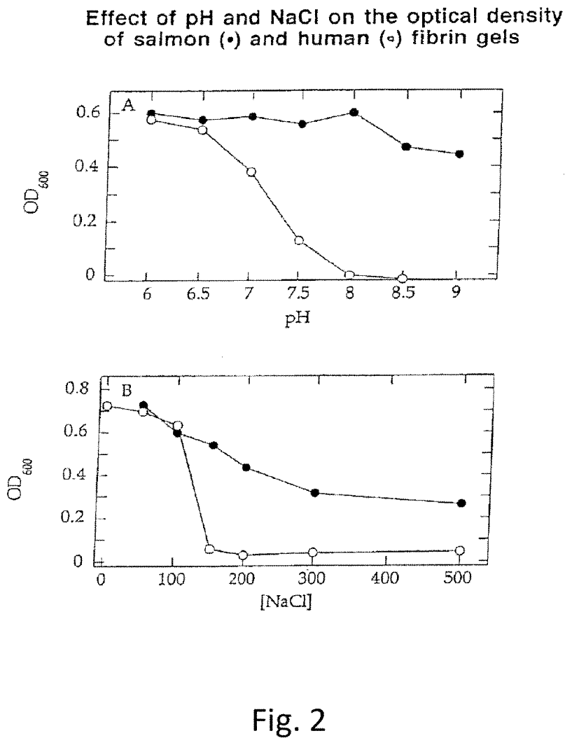

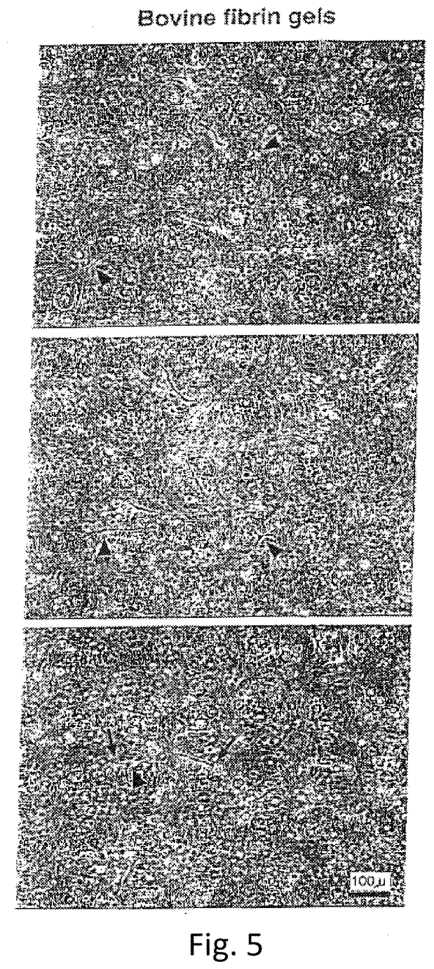

Advantages of the present invention include the following:
Salmonid plasma components are unlikely to transmit mammalian infections agents. The wide evolutionary distance between fish and mammals, and the differences in body temperature between mammals and the cold-water fishes such as trout and salmon, provide safety from cross-species infection.
Salmonid plasma components are more effective than mammalian products for certain tissue culture applications. Because salmon lipids and plasma proteins must function in vivo over a wide range of temperature, pH, and osmolality, their performance in tissue culture reflects these properties. Salmon lipids are highly unsaturated and rich in omega-3 fatty acids. Lyopholized salmon fibrinogen is easily reconstituted at room temperature, unlike lyophilized mammalian fibrinogens, which must be heated to 37° C. (Catalog 1999, Calbiochem, San Diego, Calif.). Gels produced with salmon fibrinogen and thrombin are more resistant to changes in pH and NaCl concentration than gels made with human proteins (FIG. 2). Mammalian neurons grown in salmon gels show enhanced process outgrowths compared to neurons grown in mammalian gels (FIGS. 5-7).
Salmonid plasma components can be produced with lot-to-lot consistency. An important requirement is for donor fish to be reared under consistent and reproducible conditions, not necessarily the nature or specifics of these conditions. The reproducibility of conditions reduces variability in quantity and quality of plasma components.
The physiology of fishes, including plasma composition, is regulated to a much greater degree by external factors than that of mammals. Therefore, plasma composition can be manipulated by environmental or nutritional means not possible in mammals. For example, amounts of cholesterol and high-density lipoprotein (HDL) are significantly different in salmon held at different salinities or fed different diets. (Rabin and Vernier, 1989).

According to the invention, the culture of representative mammalian tissue has been demonstrated. The plasma components used were lipids, fibrinogen, and thrombin from the plasma of Atlantic salmon (S. salar). This species was used for the disclosed examples because consistent and reproducible methods for their production are well established, large numbers are reared in commercial aquaculture operations, and individual fish are large enough for blood to be obtained easily. These particular plasma components were chosen because they are plasma fractions frequently used for mammalian cell culture, and serve as examples of other fish plasma components, such as transferrin, fibronectin, and enzymes, which can also be similarly useful.

### Preparation and Extraction

The process begins with the consistent and reproducible conditions under which donor fish are reared. All fish used as plasma sources preferably are progeny of domesticated broodstock, inspected for fish disease according to the American Fisheries Society “Blue Book” standards, preferably larger than two kilograms, and reared by standard husbandry methods. They can be, but do not necessarily have to be, sexually immature, in the log-phase of growth, and fed a commercially pelleted food appropriate to the species.

Water temperature at the time of bleeding is preferably 4° C. to 12° C. The fish are preferably starved for five days before bleeding to reduce proteolytic enzymes and non-protein nitrogen. Each fish is stunned, such as by a blow to the head, or by immersion in ice-water, or in water containing CO₂ or other fish anesthetic, in order to render the fish to a level of loss of reflex activity (unconsciousness) as defined by Schreck and Moyle, (1990). Whole blood is then drawn, preferably from the caudal artery or vein with a sterile needle and a syringe or vacuum device containing an anticoagulant such as ACD (acid citrate dextrose), trisodiun citrate, or other anticoagulant commonly used in human blood-banking.

Whole blood is held for no more than four hours at 2°-4° C., centrifuged at 2°-4° C., and then the plasma is frozen, preferably at ˜60° C. or lower. Freezing prevents degradation of plasma proteins, especially in long term storage, and improves protein yield. The frozen plasma can be thawed before extracting components as needed.

Because of the large amounts of highly unsaturated fatty acids, plasma to be used for lipid extraction preferably is handled under argon, or an antioxidant such as alphatocopherol, BHT, or mercaptoethanol at less than 1 ppm is added, and then is frozen, for example at −80° C. For plasma lipids, an extraction procedure (for example, that described in detail by Condie, 1979, or Ando, 1996) is applied to whole plasma. In summary, this process utilizes fumed silica to adsorb the lipids from the plasma fraction.

Lipids are eluted from the silica with sodium citrate at pH 10-11 and dialyzed against a saline solution, and additional antioxidants (for example, ascorbic acid, BHA, BHT) are added. The lipid is then analyzed for cholesterol content and concentrated to a level of 5 to 15 mgs/ml cholesterol. The lipid is then stored under vacuum or argon at −80° C.

For fibrinogen extraction and purification, the method of Silver et al., 1995 preferably is used. This method is based on ammonium sulfate precipitations, which yields greater than 95% pure fibrinogen (by SDS-PAGE). Preferably, thrombin is prepared by the method of Ngai and Chang, 1991.

### EXAMPLE #1

### Fish Lipids in Tissue Culture

A green monkey kidney cell line (Vero) commonly used in commercial culture, the Promega Nonradioactive Cell Proliferation Assay (Fisher Healthcare, Houston, Tex.), and serum-free media, VP-SFM (Life Technologies, Inc., Grand Island, N.Y.), were used to evaluate the fish lipid component.

Test media were formulated as follows: 1. Control 2. VP-SFM only 3. VP-SFM plus salmon lipid (0.25 mgs/L cholesterol) 4. VP-SFM plus salmon lipid (1.0 mgs/L cholesterol) 5. VP-SFM plus salmon lipid (5.0 mgs/L cholesterol).

The frozen fish lipid was thawed in a water bath at 2-4° C. Assays were conducted using 24-well polystyrene culture plates. Each well was seeded with 30,000 cells in VP-SFM medium containing 5% fetal calf serum (FBS). The cells were allowed to attach and spread for a 24-hour period, and then the growth medium was removed by aspiration. All wells were rinsed thoroughly with the VP-SFM medium and the test formulations (3 wells each) were added.

The cells were then incubated at 37° C. for 48 hours in a 5% CO₂ atmosphere in 95% relative humidity.

After 48 hours, the cultures were examined and quantified using the Promega Nonradioactive Cell Proliferation Assay. This assay measures viable cells only and is based on a standard curve of cell concentrations determined for each cell type. Results for each condition were averaged and statistically compared using ANOVA (one-way analysis of variance).

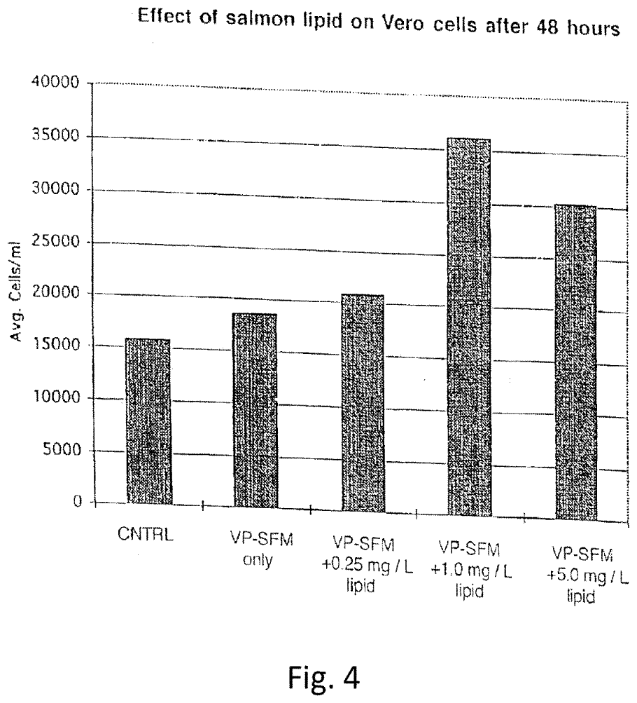

There was no significant difference between the number of viable cells in the VP-SFM and the VP-SFM plus the lower concentration of salmon lipid, showing that the fish material was not toxic. However, addition of salmon plasma lipid at the higher concentration to the media (VP-SFM plus 1.0 mgs/L cholesterol) enhanced growth significantly (P=<0.001). The highest concentration of salmon lipid (5.0 mgs/L) was less effective (FIG. 4).

These results show that the salmon plasma lipids enhance the growth of a mammalian cell line (Vero) in culture.

### EXAMPLE #2

### Neurons in Fish Fibrin Gels

Growing mammalian neurons in a gel made from fish plasma components is an example of in vitro cell culture with potential in vivo (tissue engineering) applications. Cell survival and neurite process extension in gels are established models for nerve regeneration in vivo (Schense et al., 2000).

Primary spinal cord neuronal cultures were prepared as described by Dunham (1988) from embryos harvested from timed-pregnant female mice (C57BL/6J; Jackson Laboratory, Bar Harbor, Me.). Culture media and conditions for the neurons were also as described by Dunham (1988).

Lyophilized salmon fibrinogen and thrombin were reconstituted in water at room temperature, and the gels were prepared by treating 3 mg/L salmon fibrinogen with 1.5 U/ml salmon thrombin and adding 1.4 mM calcium in cell culture media. Similar gels were prepared using lyophilized human and bovine fibrinogen and thrombin. In order to embed neurons in the gel, fibrinogen, neurons, and cell culture media were mixed together, and then thrombin was added. The solution was mixed gently 2-3 times and transferred to a polylysine-coated coverslip. The formation of the fish fibrin gels was similar to gels formed from mammalian material and resulted in a solid gel within 30 minutes at room temperature with a shear modulus of about 550 dynes/cm. After at least 30 minutes, the gels were covered with neuronal cell culture media and placed in a 37° C. cell culture incubator.

The neurons in the fish and mammalian fibrin gels were viewed on a Nikon Diaphot 300 inverted microscope, and images were captured with a Micromax cooled CCD camera driven by Inovision image processing software on a SGI 02 computer. Images were processed and compiled using Adobe Photoshop 5.0. Neurite length was quantified using NIH Image, and all data was analyzed using Kaleidagraph.

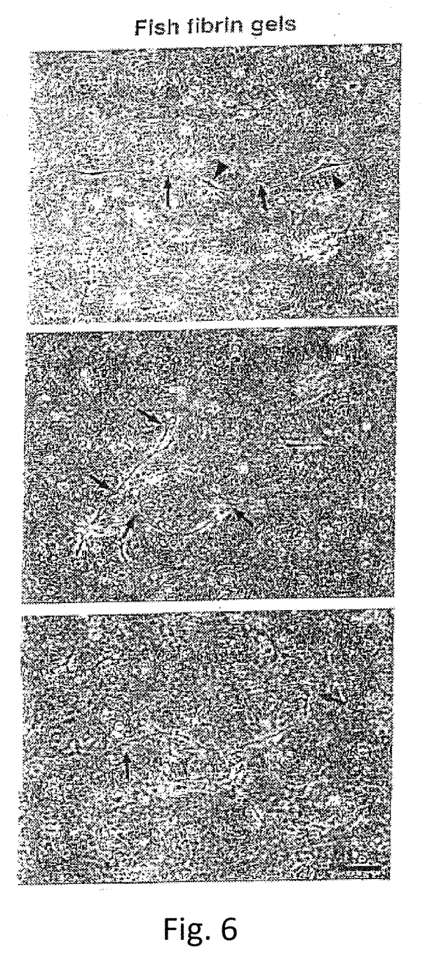

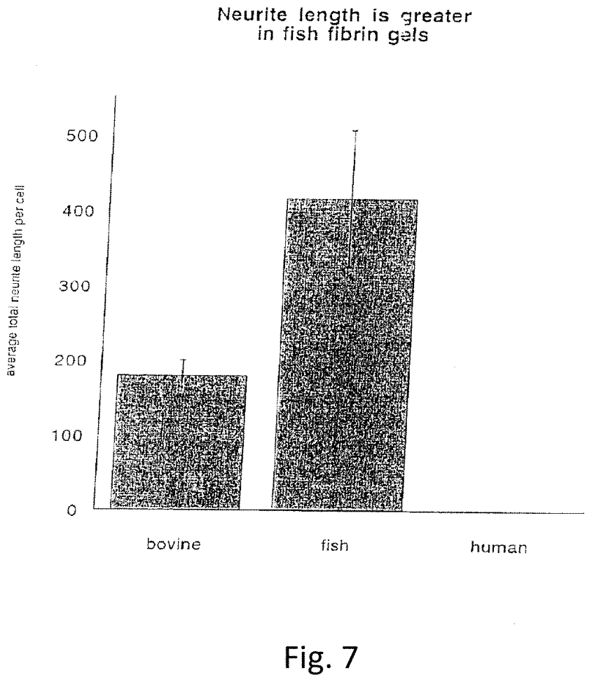

After two days in culture, human fibrin gels began to disintegrate, and by day 4, the gel was completely digested away, leaving only sparse cells attached to the glass. In contrast, bovine and fish gels remained intact for at least a week. FIGS. 5 and 6 show several examples of neuronal cell bodies (arrowheads) and extended processes (arrows) in the gels. Fish fibrin gels contained multiple neurons with processes longer than those of the neurons in the bovine gels, and extending in three dimensions into the gel. Quantitation of neurite length (microns) in fish gels compared to that in bovine gels reveals that neurite length in fish gels is greater by a factor of 2.3 (fish gels=416.25.+−.89.9 sem, n=10 cells: bovine gels=179.18.+−.20.9 sem, n=8 cells) (FIG. 7).

These results show a clear and significant enhancement of neurite length for mammalian spinal cord neurons when they are cultured in a salmon fibrin gel instead of the mammalian gel.

These experiments demonstrate that those with ordinary skill in the field of tissue culture can substitute fish plasma components for the mammalian plasma substances now used for mammalian tissue culture, and realize significant advantages from the fish material that were not provided by fish whole plasma and serum products. For example, human stem cells have in common the ability to self-renew and differentiate into multiple unique cell types. Recent studies indicate that embryonic, hematopoetic, and neural stem cells share many molecular markers that, as in the case of neural and embryonic stem cells, make them more like each other than like the tissues they differentiate into (Ramalho-Santos et al., 2002; Ivanova et al., 2002). Differentiated cells also often have many characteristics in common despite their diverse functions. For example, cells from organs as disparate as the brain and the pancreas benefit from growth in a deformable three-dimensional matrix such as fibrin (Flanagan et al., 2002; Beattie et al., 2002).

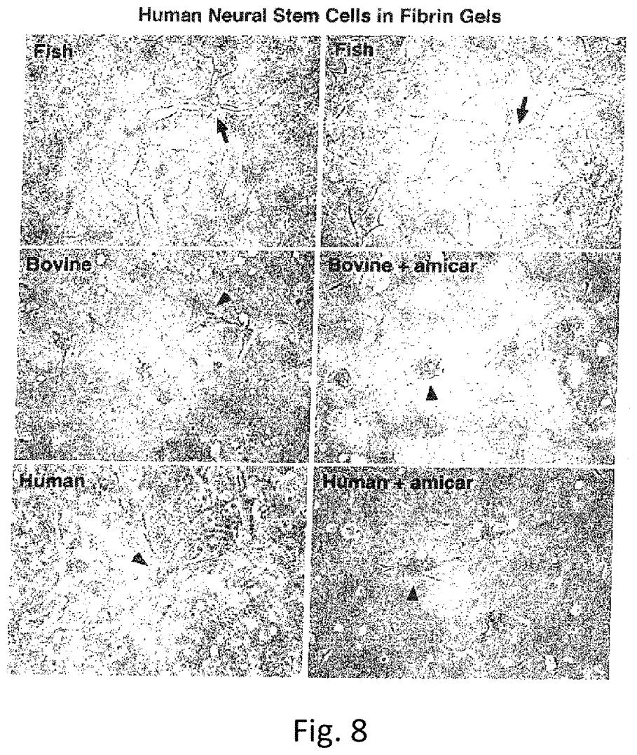

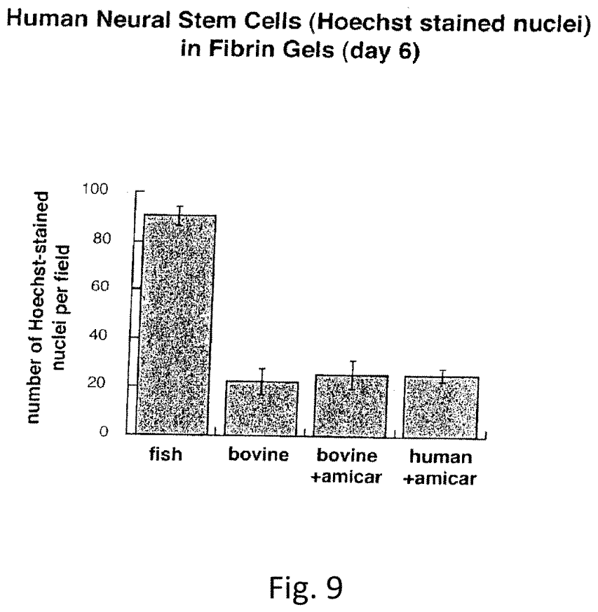

FIG. 8 shows several examples of human neural stem cells in fibrin gels, including fish, bovine, and human fibrin gels. FIG. 9 graphs the number of Hoechst-stained human neural stem cell nuclei present in the fibrin gels after six days, for each of four different fibrin gels. As shown, the number of nuclei per field present in the fish gels were far greater than those present in the non-fish gels.

These extraction techniques are illustrative of those currently in use, but other techniques may be equally effective. The essential requirements are that there must be no cytotoxic chemical residues in the product, and plasma lipids must be protected from oxidation. The optimum practice for extracting lipids requires that all process temperatures must remain below 4° C. Otherwise, however, for thawing frozen plasma destined for extraction of proteins such as fibrinogen, this practice often results in the formation of cryoprecipitate, a concentration of cold-insoluble high molecular weight proteins (especially fibrinogen) that occurs during thawing. Much of the fibrinogen in this case would be then lost in the remaining plasma. Thus, briefly raising the plasma thawing temperature closer to 5-6° C. allows the fibrinogen to go back into solution and process yields improve.

Further, the plasma, or the extracted plasma component, should be tested for endotoxin, a common cytotoxic chemical contaminant. Even very low levels of endotoxin are cytotoxic, and therefore a maximum threshold for each application should be established, and only plasma or components having an endotoxin level below this threshold should be used, although the actual threshold level applied can be lower or higher based on the particular application or end-use. Testing for this toxic chemical, for example using the Limulus Amebocyte Lysate or similar assay, could prevent failure of salmon proteins and lipids to grow and maintain mammalian cells.

Endotoxin testing can be performed on fresh or frozen plasma or on the end product extracted from the plasma. Predetermined acceptable limits vary widely, and depend on such factors as degree of processing, dilution factor, end-product application, and type of cells used. For example, products similar to salmon plasma include a bovine serum with 10 EU/ml sold by Thermofisher Scientific Co and often used for in vitro diagnostics. As another example, the FDA sets limits for certain products such as Water for Injection (0.25 EU/ml), and for recombinant proteins (1 EU/ml) (Schwartz). In addition, Corning Discovery Labware, Inc. produces “Matrigel”, a Matrix for 3D cell-culture similar to a fibrin gel made with salmon fibrinogen and thrombin. A typical endotoxin level as reported in a Matrigel Certificate of Analysis is 3.69 EU/ml.

Overall, for each production lot of each salmon protein the acceptable endotoxin level may vary, but it must be known and reported on a Certificate of Analysis.

Additional aspects of the plasma component should be taken into account as well. For example, cells respond to both chemical and mechanical stimuli, and when a salmon fibrin gel composed of fibrinogen and thrombin is used as a substrate for mammalian cell culture, the mechanical properties of that gel are important. Rheometry of a fibrin gel is necessary to determine the property of sheer modulus or stiffness as measured in for example, Pascals.

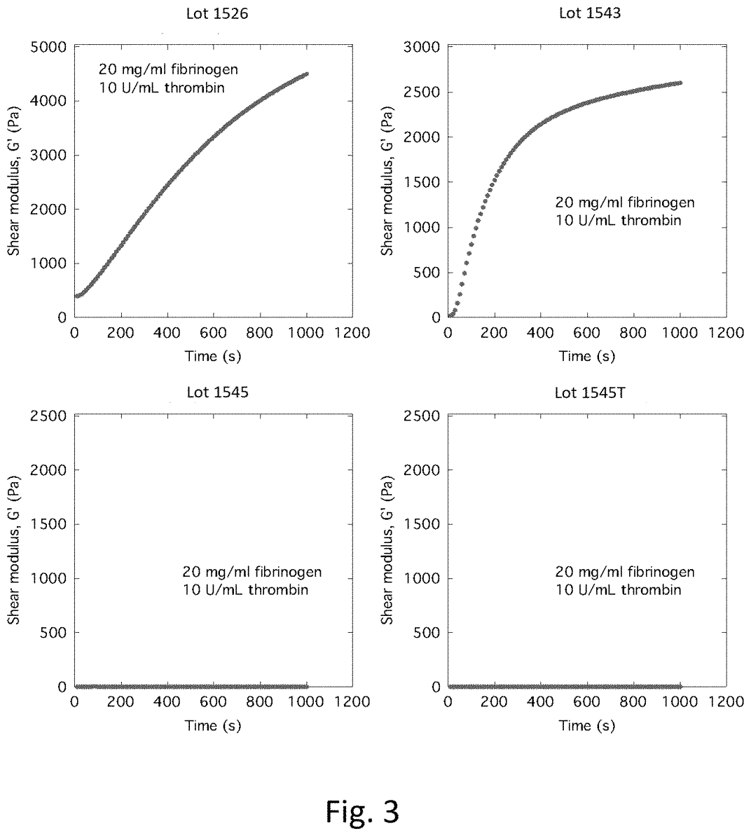

Sawyer et al., 2003 and 2005 do not mention the need to test the sheer modulus or stiffness of a fibrin gel composed of salmon fibrinogen and thrombin. For many applications, this property should exceed 1000 Pascals (Pa) to allow dilution, but this measure of clot stiffness is variable in the culture of mammalian tissue. For example, Georges et al., 2006 found that neurons prefer a soft (˜50 Pa) salmon fibrin gel, and astrocyte spread and adherence is greater on stiffer gels. Therefore, the stiffness of a particular gel relates to its suitability for use in certain applications. FIG. 3 shows variation in stiffness of several lots of salmon gels formed by polymerizing salmon fibrinogen with salmon thrombin including unusable gels made with two different lots of fibrinogen (#1545 and 1545T). Because all the gels in FIG. 3 polymerize and appear normal, the failing gels could not be detected without rheometry.

Failure of a salmon fibrin gel relates to the batch or lot of fibrinogen used, and can be caused by many factors including the health status of the fish, process temperatures above 6° C., partial activation of proteases, and many unknown factors. Therefore, rheometry is important before salmon fibrin gels can be used as a scaffold or substrate in mammalian cell or tissue culture.

Thus, in general according to the invention, a process of using a fish plasma component in a nutrient medium for cell culture includes obtaining a fish that is a progeny of domesticated broodstock that are reared under consistent and reproducible conditions. Blood is obtained from the fish, and plasma is separated from the blood. One or more specific components of the plasma are then extracted, and cells are cultured in a nutrient medium using the one or more extracted plasma components, and none of any remainder of the plasma. At any point, the blood, the plasma, and/or the plasma components can be tested for the presence and/or a level of endotoxin, and the process is only continued if the testing indicates an endotoxin level below a predetermined threshold. This threshold level can be predetermined based on a general standard or on the particular end-use application intended for the plasma component. The cells cultured using the extracted one or more plasma components are other than fish cells.

The fish from which the blood is obtained preferably is larger than two kilograms and/or reared by standard husbandry methods. The fish can be a cold-water fish, such as, for example, a salmonid. Blood can be obtained from the fish by, for example, rendering the fish to a level of loss of reflex activity and drawn, for example, from a caudal blood vessel. The levels of proteolytic enzymes and non-protein nitrogen present in the blood of the fish can be reduced prior to rendering the fish to a level of loss of reflex activity.

The process can also include freezing the plasma for storage and thawing the plasma for further processing or use. For example, the plasma can be frozen at a temperature of −60° C. or lower after separating the plasma from the blood, and thawed before extracting one or more specific components of the thawed plasma. Thawing the frozen plasma can include allowing the frozen plasma to reach a temperature at which any cryoprecipitate present in the plasma redissolves back into solution, at a maximum of 6° C.

Different components of the plasma can be of interest for use according to the process of the invention, depending on the end-use application. For example, the one or more specific components of the plasma can include fibrinogen and thrombin, and a gel can be prepared with the fibrinogen, the thrombin, and calcium in tissue culture media. The cells can then be cultured by embedding the cells in the gel. Preferably, a sheer modulus of the gel is determined, such as by performing rheometry on the gel. The cells cultured using the extracted one or more plasma components can be determined based at least in part on the determined sheer modulus of the gel.

The one or more components extracted from the plasma can include lipids, and an antioxidant can be added to the plasma prior to extracting the one or more components from the plasma. A plasma protein, such as, for example, transferrin, albumin, fibrinogen, fibronectin, thrombin, and/or enzymes, can also be extracted.

The plasma can be separated from the blood by centrifuging the blood, and the one or more components should be extracted from the plasma such that no cytotoxic chemical residues remain in the one or more plasma components, and no oxidation of plasma lipids occurs.

Culturing cells in the one or more extracted plasma components can include seeding the cells in a defined medium and the one or more extracted plasma components.

The cells cultured using the one or more extracted plasma components can be, for example, mammalian cells, for example, neurons and/or stem cells. The cells cultured using the one or more extracted plasma components can include organ tissue. The cells cultured using the one or more extracted plasma components can be insect cells.

Preferred and alternative embodiments have been described in detail. It is contemplated, however, that various modifications of the disclosed embodiments fall within the spirit and scope of the invention. The scope of the appended claims, therefore should be interpreted to include such modifications, and is not limited to the particular embodiments disclosed herein. For example, the use of these and other fish plasma components in mammalian tissue culture, or fish plasma components in insect cell culture, especially in the production of recombinant proteins, is a contemplated aspect of the present invention to satisfy the same objects and provide the same advantages as those for mammalian cell culture.

### REFERENCES

Ando, S. 1996. Adsorption of Serum Lipoproteins from Rainbow Trout to Dextran Sulfate Cellulose. Fisheries Science 62 (3): 410-415.
Babin, P. J. and Vernier, J. 1989. Plasma Lipoproteins in Fish. J. Lipid Research 30:467-489.
Cohn, E. J., Strong, L. E., Hughs, W. L., Mulford, D. J., Ashworth, J. N., Melin, M., Taylor, H. L. 1946. Preparation and Properties of Serum and Plasma Proteins IV. A System of Separation Into Fractions of the Protein and Lipoprotein Components of Biological Tissues and Fluids. J. Am. Chem. Soc. 68:459-475.
Condie, R. M. 1979. Preparation of Intravenous Human and Animal Gamma Globulins and Isolation of Albumin. U.S. Pat. No. 4,136,094.
Davidson, W. S., Bartlett, S. E., Birt, T. P., Birt, V. L., Green, J. M. 1989. Identification and Purification of Serum Albumin From Rainbow Trout (Salmo gairdneri). Comp. Biochem. Physiol. 93B (1):5-9.
DeKoning, J., and Kattari, S. 1992. An Improved Lymphocyte Culture Medium Incorporating Plasma for in vitro Antibody Production and Mitogenesis. Fish and Shellfish Immunology 2:275-285.
Denovan-Wright, E. M., Ramsey, N. B., McConnick, C. J., Lazier, C. B., Wright, J. M. 1996. Nucleotide Sequence of Transferrin cDNAs and Tissue Specific Expression of the Transferrin Gene in Atlantic Cod (Gadus Morhua). Compo Biochem: Physiol. 11:3B (2): 269-273.
Doolittle, R. M. 1987. The Evolution of the Vertebrate Plasma Proteins. Biol. Bull. 269-283.
Durham, H. D. 1988. Aggregation of Intermediate Filaments by 2,5-Hexanedione; Comparison of Effects on Neurofilaments, GFAP-Filaments and Vimentin-Filaments in Dissociated Cultures of Mouse Spinal Cord-Dorsal Root Ganglia. J. Neuropathal Exp. Denral. 47(4), 432-442.
Flanagan et al., 2002. Neurite Branching on Deformable Substrates. Neuroreport Dec. 20; 13(18):2411-15.
Georges PC et al., 2006. Matrices with compliance comparable to that of brain tissue select neuronal over glial growth in mixed cortical cultures. Biophysical J. 90 (3012-3018).
Hewlett, G. 1991. Strategies for Optimising Serum-Free Media. Cytotechnology 5:3-14.
Mylllyla G. 1991. Whole blood and plasma procurement and the impact of plasmapheresis. In: Blood Separation and Plasma Fractionation, J R Harris Editor. Wiley-Liss Inc. New York.
Ngai, P. K., and Chang, J. Y., 1991. A Novel One-Step Purification of Human Thrombin After Direct Activation of Crude Prothrombin Enriched From Plasma. Biochem J. 280:805-808.
Ramalho-Santos et al., 2002. “Sternness”: Transcriptional Profiling of Embryonic and Adult Stem Cells. Science Oct. 18; 298(5593):597-600.
Rock G. 1919. Purification of Factor VIII procoagulant from plasma. In: Blood Separation and Plasma Fractionation, J R Harris Editor. Wiley-Liss Inc. New York.
Rose, E. et al. 1990. U.S. Pat. No. 4,928,603. May 29, 1990. Method of preparing a cryoprecipitated suspension and use thereof.
Sawyer E S et al. 2003 U.S. Pat. No. 6,8599,740 B2. Jul. 29, 2003. Method of using fish plasma components for tissue culture.
Sawyer E S et al. 2005. U.S. Pat. No. 6,861,255 B1. Mar. 1, 2005. Method of using fish plasma components for tissue culture.
Schense, J. C., Bloch, J., Aebischer, P., and Hubbell, J. A., 2000. Enzymatic Incorporation of Bioactive Peptides into Fibrin Matrices Enhances Neurite Extension. Nature Biotechnology 18:415-419.
Sharpe K G et al. 2012. Salmon fibrin treatment of spinal cord injury promotes functional recovery and density of serotonergic innervation. Exp. Neurol. 235 (1) 345-56.
Schreck, C. B. and Moyle, P. B. 1990. Methods for Fish Biology, Am. Fish. Soc. Bethesda, Md. Silver, F. R., Wang, M. C., and Pins, G. D. 1995. Preparation of Fibrin Glue: A Study of Chemical and Physical Methods. J. Appl. Biomaterials 6(3):175-183.
Silver, F. R. Wang, M. C. and Pins, G. D. 1995. Preparation of Fibrin Glue: A Study of Chemical and Physical Methods. J. Appl. Biomaterials 6(3):175-183.
Smith J R et al. Salmon and human thrombin differentially regulate radicular pain, glial induced inflammation, and spinal neuronal excitability through proteus-activated receptor-1. PLoS 1 8(11)e80006.
Swartz H et al. 2014. Residual endotoxin contaminations in recombinant proteins are sufficient to activate CD1c+dendritic cells. PLoS 1 2014 9(12): e113840.
Zhu et al., 2017. Selective expansion of skeletal muscle stem cells in soft three-dimensional fibrin gels. Stem Cells Translational Medicine 2017 6: 1412-1423.
FDA Guidance for Industry: Bacterial Endotoxin/Pyrogens, #40, June 2012
Thermofisher Scientific Inc. Pittsburgh, Pa. Catalog number 26-140-095
Corning Discovery Labware, Inc. Bedford Mass. 01730. Catalog number 356255

1. A process of using a fish plasma component in a nutrient medium for cell culture, comprising:
obtaining a fish that is a progeny of domesticated broodstock that are reared under consistent and reproducible conditions;
obtaining blood from the fish;
separating plasma from the blood;
extracting one or more specific components of the plasma; and
culturing cells in a nutrient medium using the one or more extracted plasma components, and none of any remainder of the plasma;
further comprising at least one of:
testing the plasma for endotoxin level prior to extracting the one or more specific components of the plasma,
testing the plasma for endotoxin level prior to culturing the cells in the nutrient medium using the one or more extracted plasma components, and
testing the one or more extracted plasma components for endotoxin level prior to culturing the cells in the nutrient medium using the one or more extracted plasma components;
wherein at least one of extracting the one or more specific components of the plasma, and culturing the cells is only performed if the testing indicates an endotoxin level below a predetermined threshold; and
wherein the cells cultured using the extracted one or more plasma components are other than fish cells.

2. The process of claim 1, further comprising:
freezing the plasma at a temperature of −60° C. or lower after separating the plasma from the blood; and
thawing the frozen plasma before extracting one or more specific components of the thawed plasma.

3. The process of claim 2, wherein thawing the frozen plasma includes allowing the frozen plasma to reach a temperature at which any cryoprecipitate present in the plasma redissolves back into solution.

4. The process of claim 1, wherein the one or more specific components of the plasma include fibrinogen and thrombin, and culturing cells using the extracted plasma components, and none of any remainder of the plasma, in a nutrient medium, includes preparing a gel with the fibrinogen, the thrombin, and calcium in tissue culture media.

5. The process of claim 4, wherein culturing the cells further includes embedding the cells in the gel.

6. The process of claim 4, further comprising determining a sheer modulus of the gel.

7. The process of claim 6, wherein determining the sheer modulus of the gel includes performing rheometry on the gel.

8. The process of claim 7, further comprising selecting the cells cultured using the extracted one or more plasma components based at least in part on the determined sheer modulus of the gel.

9. The process of claim 1, wherein the fish from which the blood is obtained is at least one of larger than two kilograms and reared by standard husbandry methods.

10. The process of claim 1, wherein obtaining blood from the fish includes rendering the fish to a level of loss of reflex activity.

11. The process of claim 10, further comprising drawing blood from a caudal blood vessel.

12. The process of claim 10, wherein obtaining blood from the fish further includes, prior to rendering the fish to a level of loss of reflex activity, reducing the levels of proteolytic enzymes and non-protein nitrogen present in the blood of the fish.

13. The process of claim 1, wherein separating plasma from the blood includes centrifuging the blood.

14. The process of claim 1, wherein extracting the one or more components from the plasma includes performing an extraction process on the plasma such that no cytotoxic chemical residues remain in the one or more plasma components, and no oxidation of plasma lipids occurs.

15. The process of claim 1, wherein the cells cultured using the one or more extracted plasma components are mammalian cells.

16. The process of claim 15, wherein the mammalian cells are neurons.

17. The process of claim 15, wherein the mammalian cells include stem cells.

18. The process of claim 1, wherein the cells cultured using the one or more extracted plasma components include organ tissue.

19. The process of claim 1, wherein the cells cultured using the one or more extracted plasma components are insect cells.

20. The process of claim 1, wherein the fish is a cold-water fish.

21. The process of claim 20, wherein the fish is a salmonid.

22. The process of claim 1, wherein culturing cells in the one or more extracted plasma components includes seeding the cells in a defined medium and the one or more extracted plasma components.

23. The process of claim 1, wherein the one or more components extracted from the plasma include lipids.

24. The process of claim 23, further comprising adding an antioxidant to the plasma prior to extracting the one or more components from the plasma.

25. The process of claim 1, wherein the one or more components extracted from the plasma include a plasma protein.

26. The process of claim 25, wherein the plasma protein includes any one or more of transferrin, albumin, fibrinogen, fibronectin, thrombin, and enzymes.
